(19) United States
(12) Patent Application Publication
(10) Pub. No.: US 2026/0256507 A1
(43) Pub. Date: Sep. 3, 2026

(54) COMPOSITIONS OF AND METHODS FOR A COLD SLURRY HAVING HYALURONIC ACID

(71) Applicants: Sameer SABIR, Arlington, MA (US); Charles SIDOTI, Milton, MA (US); Olivier KAGAN, Belmont, MA (US); Mansoor M. AMIJI, Attleboro, MA (US); Joseph AARON, Cambridge, MA (US); Danielle BRUCATO, Cambridge, MA (US); Jun LI, Cambridge, MA (US)

(72) Inventors: Sameer SABIR, Arlington, MA (US); Charles SIDOTI, Milton, MA (US); Olivier KAGAN, Belmont, MA (US); Mansoor M. AMIJI, Attleboro, MA (US); Joseph AARON, Cambridge, MA (US); Danielle BRUCATO, Cambridge, MA (US); Jun LI, Cambridge, MA (US)

(73) Assignee: Brixton Biosciences, Inc., Cambridge, MA (US)

(21) Appl. No.: 18/837,879

(22) PCT Filed: Feb. 10, 2023
(86) PCT No.: PCT/US2023/062443

§ 371 Date: Aug. 12, 2024

Related U.S. Application Data
(60) Provisional application No. 63/309,291, filed on Feb. 11, 2022.

(51) Int. Cl. A61B 18/02; A61K 9/00; A61K 31/728; A61K 47/02; A61K 47/10; A61K 47/24; A61K 47/69

(52) U.S. Cl. CPC A61B 18/02; A61K 9/0019; A61K 31/728; A61K 47/02; A61K 47/10; A61K 47/24; A61K 47/6907; A61B 2018/0293

(57) ABSTRACT

Disclosed herein is a composition comprising an amount of water; a hyaluronic acid; and a first excipient, wherein the composition is configured to be formed into a ‘flowable cold slum’ when the composition is exposed, to a temperature of 0° C. or less.

### TECHNICAL FIELD

This application claims priority under 35 U.S.C. § 119(c) to U.S. Ser. No. 63/309,291, filed Feb. 11, 2022, the contents of which are hereby incorporated by reference in its entirety.

The present disclosure relates generally to compositions and methods for manufacturing biomaterials that form flowable and/or injectable cold slurries. More particularly, the present invention preferably relates to a formulation of a biocompatible solution that contains a liquid (e.g., saline, water, or phosphate-buffered saline), glycerol, hyaluronic acid, a poloxamer, and, optionally, lipids.

### BACKGROUND

Cold slurries (e.g., ice slurries) are known in the art as compositions that are made of sterile ice particles of water, varying amounts of excipients or additives such as freezing point depressants, hydrotropic molecules, and, optionally, one or more active pharmaceutical ingredients, as described in U.S. application Ser. No. 15/505,042 (“'042 Application”; Publication No. US2017/0274011), the disclosure relating to the formulation of cold slurry compositions is incorporated by reference in its entirety herein. Prior art cold slurries can be delivered, preferably via injection, to a tissue of a subject, preferably a human patient, to cause selective or non-selective cryotherapy and/or cryolipolysis for prophylactic, therapeutic, or aesthetic purposes. Injectable cold slurries may be used for treatment of various disorders that require inhibition of nerve conduction. For example, U.S. application Ser. No. 15/505,039 (“'039 Application”; Publication No. US2017/0274078), the disclosure relating to the reversible inhibition of nerve conduction is incorporated by reference in its entirety herein, discloses the use of slurries to induce reversible degeneration of nerves (e.g., through Wallerian degeneration) by causing crystallization of lipids in the myelin sheath of nerves. The '039 Application also discloses using injectable cold slurries to treat various other disorders that require inhibition of somatic or autonomic nerves, including motor spasms, hypertension, hyperhidrosis, and urinary incontinence.

A method of preparing a cold slurry is shown in U.S. application Ser. No. 16/080,092 (“'092 Application”; Publication No. US2019/0053939), incorporated by reference in its entirety herein. However, the method disclosed in the '092 Application requires the point of care to manufacture the cold slurry by installing medical ice slurry production system. This technique also requires the point of care to take steps to maintain sterility of the cold slurry during manufacture and prior to administration. Alternative methods of preparing a cold slurry are disclosed in U.S. Pat. No. 11,241,330 and International Publication Number WO 2022/261494 A1 (“'494 PCT”). The disclosure in WO 2022/211904 A1 (“'904 PCT”) pertaining to methods of manufacture is incorporated by reference herein. The disclosure in the '494 PCT pertaining to cold slurry compositions is incorporated by reference herein. The disclosure in the present application is compatible with the methods and systems disclosed in the International Publication No. WO 2017/147367 A1 (“'367 PCT”) and the '904 PCT.

The '904 PCT discloses a method of easily transporting a sterile biomaterial to a point of care using standard shipping techniques, where the biomaterial can be transformed into a flowable and injectable cold slurry at a point of care without requiring manufacturing equipment to be available at the point of care and without compromising the sterility of the biomaterial at the point of care. The disclosure in the present application is compatible with the methods and systems disclosed in the '904 PCT.

There exists a need for compositions and methods that allow for simple transport, storage, and preparation of a flowable and/or injectable cold slurry at a clinical point of care without compromising the sterility of the biomaterial (e.g., the solution that will be transformed into the cold slurry) during preparation, without requiring specialized manufacturing equipment to be available at the point of care, and without compromising the sterility of the biomaterial at the point of care. The present disclosure addresses this need by providing for improved cold slurry compositions and methods of preparation that allow for a biocompatible solution to be received at a point of care in an easily shipped and stored container that the point of care can place into a standard freezer and, optionally, perform further physical agitation of the container's internal contents to transform the biocompatible solution into a therapeutic substance, e.g., a flowable and/or injectable cold slurry. The present disclosure describes a composition and methods that can provide an adequate and consistent amount of ice particles after being exposed to freezing temperatures, and reliably allow injection of cold slurry through syringe needles.

### SUMMARY

In certain aspects, the present disclosure provides a composition comprising an amount of water, a hyaluronic acid, and a first excipient, wherein the composition is configured to be formed into a flowable cold slurry comprising a plurality of ice crystals when the composition is exposed to a temperature of 0° C. or less.

In certain embodiments, the composition further comprises a water-soluble surfactant. In further embodiments, the water-soluble surfactant is a poloxamer molecule. In certain embodiments, wherein the composition comprises a plurality of poloxamer molecules. In certain embodiments, the composition comprises a poloxamer particle, and wherein the poloxamer particle comprises a plurality of poloxamer molecules. In further embodiments, the poloxamer particle is a micelle. In certain embodiments, the poloxamer molecule is selected from the group consisting of poloxamer 101, poloxamer 105, poloxamer 108, poloxamer 122, poloxamer 123, poloxamer 124, poloxamer 181, poloxamer 183, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, poloxamer 182 dibenzoate, and a combination thereof. In certain embodiments, poloxamer is poloxamer 407, and wherein the concentration of the poloxamer 407 is between about 0.1% (w/w) and 10% (w/w). In certain embodiments, the concentration of the poloxamer 407 in the composition is about 0.5% (w/w). As used herein, (w/w) and (w/v) are interchangeable.

In certain embodiments, the composition further comprises a first excipient selected from the group consisting of a salt, an ion, Lactated Ringer's solution, a sugar, a biocompatible surfactant, a polyol, and a combination thereof.

In certain embodiments, the first excipient is glycerol. In some embodiments, a concentration of the glycerol in the composition is between about 12% and 25% (w/w). In certain embodiments, the concentration of the glycerol in the composition is about 19% (w/w).

In certain embodiments, the composition further comprises a second excipient. In some embodiments, the second excipient is sodium chloride or sodium phosphate to form saline or phosphate-buffered saline.

In certain embodiments, the composition further comprises a third excipient. In some embodiments, the third excipient is a non-water-soluble substance. In some embodiments, non-water-soluble substance is a lipid. In some embodiments, the lipid is selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-a-phosphatidylcholine (soy PC), phosphatidylethanolamine, (PE), phosphatidylserine (PS), phosphatidylglycerol (PG).

In certain aspects, the compositions disclosed herein are configured to form the plurality of ice crystals when the composition is exposed to a temperature of between about −25° C. and about −5° C.

In certain aspects, the compositions disclosed herein are configured to have an injection force of less than about 30 lbf when injected through a 16G needle, a 17G needle, an 18G needle, a 19G needle, a 20G needle, a 22G needle, a 23G needle, or a 24G needle.

In certain aspects, the compositions disclosed herein are configured to have an injection force of less than about 30 lbf when injected through a 17G needle or an 18G needle.

In certain aspects, methods of preparing a cold slurry for administration to a patient at a clinical point of care are provided. In certain embodiments, the method comprises preparing a composition comprising a hyaluronic acid and an amount of water; adding a first excipient to the composition, wherein the excipient comprises a water-soluble surfactant; wherein the composition is configured to form a cold slurry comprising a plurality of ice particles when the composition is cooled to a temperature below about 0° C.

In certain aspects, the water-soluble surfactant is a hydrotropic molecule. In certain embodiments, the hydrotropic molecule is a poloxamer molecule. In certain embodiments, the poloxamer molecule is selected from the group consisting of poloxamer 101, poloxamer 105, poloxamer 108, poloxamer 122, poloxamer 123, poloxamer 124, poloxamer 181, poloxamer 183, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, poloxamer 182 dibenzoate, and a combination thereof. In certain embodiments, the poloxamer is poloxamer 407, and wherein the concentration of the poloxamer 407 is between about 0.1% (w/w) and 10% (w/w). In certain embodiments, the concentration of the poloxamer 407 in the composition is about 0.5% (w/w).

In certain aspects, methods provided herein further comprise adding a second excipient to the composition, wherein the composition including the second excipient is configured to form the cold slurry when the composition is cooled to a temperature below about 0° C. In certain embodiments, the second excipient is selected from the group consisting of a salt, an ion, Lactated Ringer's solution, a sugar, a biocompatible surfactant, a polyol, and a combination thereof.

In certain embodiments, the second excipient is glycerol. In certain embodiments, a concentration of the glycerol in the composition is between about 12% and 25% (w/w). In certain embodiments, the concentration of the glycerol in the composition is about 19% (w/w).

In certain aspects, the methods provided herein further comprise adding a third excipient to the composition, wherein the composition including the second excipient and the third excipient is configured to form the cold slurry when the composition is cooled to a temperature below about 0° C. In certain embodiments, the third excipient is sodium chloride or sodium phosphate, to form saline or a phosphate-buffered saline.

In certain aspects, the methods provided herein further comprise adding a fourth excipient to the composition, wherein the composition including the second excipient, the third excipient, and the fourth excipient is configured to form the cold slurry when the composition is cooled to a temperature below about 0° C.

In certain embodiments, the fourth excipient is a non-water-soluble substance. In some embodiments, the non-water-soluble substance is a lipid. In some embodiments, the lipid is selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-a-phosphatidylcholine (soy PC), phosphatidylethanolamine, (PE), phosphatidylserine (PS), phosphatidylglycerol (PG).

In certain aspects, the methods provided herein further comprise a composition configured to form the plurality of ice crystals when the composition is exposed to a temperature of between about −25° C. and about −5° C.

In certain aspects, the methods provided herein comprise a composition configured to have an injection force of less than about 30 lbf when injected through a 16G needle, a 17G needle, an 18G needle, a 19G needle, a 20G needle, a 22G needle, a 23G needle, or a 24G needle. In certain embodiments, the composition is configured to have an injection force of less than about 30 lbf when injected through a 17G needle or an 18G needle.

In certain aspects, methods of preparing a cold slurry for administration to a patient at a clinical point of care are provided. In certain embodiments, the method comprises receiving a composition comprising a freezing point depressant and a hyaluronic acid; and cooling the composition to a temperature below about 0° C. to form a cold slurry, wherein the cold slurry comprises a plurality of ice particles. In certain embodiments, the freezing point depressant is glycerol.

In certain aspects, the composition further comprises an amount of a poloxamer molecule. In certain embodiments, the poloxamer molecule is selected from the group consisting of poloxamer 101, poloxamer 105, poloxamer 108, poloxamer 122, poloxamer 123, poloxamer 124, poloxamer 181, poloxamer 183, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, poloxamer 182 dibenzoate, and a combination thereof. In certain embodiments, the poloxamer is poloxamer 407, and wherein the concentration of the poloxamer 407 is between about 0.1% (w/w) and 10% (w/w). In certain embodiments, the concentration of the poloxamer 407 in the composition is about 0.5% (w/w).

In certain aspects, the receiving a composition comprises receiving the composition within a container.

In certain embodiments, the container is a first syringe. In some embodiments, the method further comprises connecting the first syringe to a second syringe, and processing the cold slurry through a back-and-forth cycle, wherein the back-and-forth cycle comprises pushing the cold slurry from the first syringe into the second syringe and pushing the cold slurry from the second syringe into the first syringe.

In certain aspects, the method further comprises processing the cold slurry through a second, a third, or a fourth back-and-forth cycle.

In certain aspects, the container is a container configured for topical application. In certain embodiments, the container configured for topical application is a first tube.

In certain aspects, the method further comprises connecting the first tube to a second tube and processing the slurry through a back-and-forth cycle, wherein the back-and-forth cycle comprises pushing the cold slurry from the first tube into the second tube and pushing the cold slurry from the second tube into the first tube.

In certain aspects, methods provided herein further comprise monitoring a temperature of the cold slurry.

In certain aspects, the monitoring comprises viewing a temperature sensitive indicator on a syringe or a container holding the cold slurry, wherein the temperature sensitive indicator is configured to indicate the temperature of the cold slurry.

In certain embodiments, the temperature sensitive indicator is a temperature sensitive sticker. In certain embodiments, the temperature sensitive indicator provides a visual indication when the cold slurry reaches a pre-determined temperature.

In certain embodiments, the pre-determined temperature is about −15° C. In certain embodiments, the pre-determined temperature is between about −19° C. and −11° C., between about −18° C. and −12° C., between about −17° C. and −13° C., or between about −16° C. and −14° C.

In certain aspects, the temperature sensitive indicator is configured to provide a visual indication when the cold slurry is at a colder temperature than the pre-determined temperature. In certain aspects, the temperature sensitive indicator is configured to provide a visual indication when the cold slurry is warmer than the pre-determined temperature.

In certain aspects, the monitoring comprises viewing a thermometer.

In certain aspects, the monitoring comprises viewing a temperature monitoring component that is embedded within a container holding the cold slurry. In certain embodiments, the temperature component is provided in the container or is provided along a fluid path.

In certain aspects, the monitoring further comprises listening for an audio indicator configured to indicate when the composition has reached a pre-determined temperature.

In certain aspects, the composition is terminally sterilized. In certain embodiments, the composition is terminally sterilized via autoclave or steam sterilization. In certain embodiments, the autoclave or steam sterilization comprises subjecting the composition to a temperature between about 118° C. and 121° C. In certain embodiments, the temperature is about 118° C.

In certain aspects, a cold slurry delivery system is provided. In certain embodiments, the cold slurry delivery system comprises a container holding a slurry composition, the container comprising a sterile barrier and a temperature indicator, wherein the container is configured to allow manual agitation of the slurry composition without breaking the sterile barrier.

In certain embodiments, the container is a syringe or a tube.

In certain aspects, the container is configured to be connected to a second container.

In certain aspects, the container and the second container are configured to be connected using a connector.

In certain aspects, the container and the second container comprise a first syringe and a second syringe, wherein the first syringe and the second syringe each comprise a male Luer component. In certain embodiments, the connector comprises a female Luer component.

In certain embodiments, the first syringe and the second syringe are connected using the female Luer component, and wherein the slurry composition is capable of being moved from the first syringe to the second syringe to manually agitate the slurry composition.

In certain aspects, a temperature sensitive indicator is provided on the container.

In certain aspects, the temperature sensitive indicator comprises a temperature sensitive sticker.

In certain aspects, the temperature sensitive indicator provides a visual indication when the slurry composition reaches a pre-determined temperature. In certain embodiments, the pre-determined temperature is about −15° C. In certain embodiments, the pre-determined temperature is between about −19° C. and −11° C., between about −18° C. and −12° C., between about −17° C. and −13° C., or between about −16° C. and −14° C.

In certain aspects, the temperature sensitive indicator provides a visual indication when the slurry composition is a colder temperature than the pre-determined temperature.

In certain aspects, the temperature sensitive indicator provides a visual indication when the slurry composition is warmer than the pre-determined temperature.

### BRIEF DESCRIPTION OF THE DRAWINGS

The following figures depict illustrative embodiments of the invention.

FIG. 1 is a table showing an exemplary formulation of a cold slurry composition described herein.

FIG. 2 is a graph showing the characterization of ice content of a cold slurry composition described herein and a cold slurry composition containing glycerol and PBS and prepared according to the methods as disclosed in, for example, the '367 PCT.

FIG. 3 depicts an embodiment of a composition that includes a plurality of poloxamer micelles, glycerol, and hyaluronic acid gel.

FIGS. 4A-B. FIG. 4A is an image depicting an exemplary configuration of two syringes to process a slurry composition in a back-and-forth process between the two syringes according to certain embodiments described herein. FIG. 4B shows a process flow diagram for preparing an injectable cold slurry using a back-and-forth (“BAF”) process by subjecting the contents to three BAF cycles according to certain embodiments described herein.

FIGS. 5A-D. FIG. 5A depicts an exemplary syringe configuration comprising a temperature sensitive indicator to measure the temperature of the syringe's contents where the indicator displays a range of temperatures according to embodiments described herein. FIG. 5B depicts an exemplary syringe configuration comprising a temperature sensitive indicator displaying a single specific temperature according to embodiments described herein. FIG. 5C depicts an exemplary syringe configuration comprising an external temperature sensitive indicator displaying “Process Now” when the contents of the syringe reach a predetermined temperature. FIG. 5D depicts an exemplary syringe configuration comprising a plurality of temperature sensitive indicators displaying messages reading “Wait”, “Process Now”, and “Discard” when the contents of the syringe reach a series of predetermined temperatures.

FIG. 6 is a graph showing injection force (lbf) to inject slurry as a function of the temperature of the freezer into which a syringe containing the composition prepared according to the present disclosure via a 17G needle.

FIGS. 7A-B. FIG. 7A is a graph showing the relationship between the equilibrium temperature (as a proxy for ice content) and the injection temperature for a composition prepared according to the present disclosure. FIG. 7B is a graph showing the relationship between the injection force (lbf) and the injection temperature for a composition prepared according to the present disclosure when injected through a 17G needle.

FIG. 8 is a graph showing the relationship between injection force (lbf) and post-processing temperature in compositions prepared according to the present disclosure and subjected to one, two, three, or four BAF cycles when injected through an 18G needle.

FIG. 9 is a plot showing injection force (lbf) versus post-processing temperature in compositions prepared according to the present disclosure that were subjected to two BAF cycles and injected either through a 17G or 18G needle.

FIG. 10 is a graph showing the characterization of ice content of compositions described herein.

FIG. 11 is a table summarizing injection force (lbf) and injection reliability for compositions described herein when prepared according to different processing methods.

FIG. 12 is a plot showing injection forces required for cold slurry compositions described herein terminally sterilized using gamma irradiation versus non-gamma irradiated compositions.

### DETAILED DESCRIPTION

The present disclosure relates generally to compositions and methods for manufacturing biomaterials that form flowable and/or injectable cold slurries. More particularly, disclosed herein is a composition comprising water, a hyaluronic acid, and at least one excipient or additive. In certain embodiments, the at least one excipient or additive is a Pluronic™ (also known as a “poloxamer”). As used herein, the term “excipient” means any substance, not itself a therapeutic agent, used as a diluent, adjuvant, and/or vehicle for delivery of a therapeutic agent (in this case the therapeutic agent is the ice) to a subject or patient, and/or a substance added to a composition to improve its handling, stability, or storage properties. The terms “excipient” and “additive” are used interchangeably herein. In some embodiments, the solution may also contain liposomes, lipids, or other lipid structures (e.g., lamellar or non-lamellar structures, bilayer and non-bilayer structures, including lipid nanoparticles, micelles, etc.), non-water-soluble substances (i.e., substances that do not dissolve in water), or a water-soluble surfactant such as a hydrotropic molecule (e.g., a polysorbate).

In some embodiments, the flowable and/or injectable or topically applied composition contains significant amounts of ice which provides therapeutic benefit for various applications. For example, therapeutic applications of cold slurry are disclosed in U.S. application Ser. Nos. 16/288,073 and 16/327,266, the disclosures related to various therapeutic applications are incorporated by reference herein.

In some embodiments, the final product to be administered via injection to a human patient or a subject (such as a human who is not a patient or a non-human animal) is a cold slurry comprised of sterile ice particles of water and varying amounts of excipients/additives, such as hyaluronic acid, a poloxamer, and/or freezing point depressants. For example, the percentage of ice particles in the cold slurry can constitute less than about 10% by weight of the slurry, between about 10% by weight and about 20% by weight, between about 20% by weight and about 30% by weight, between about 30% by weight and about 40% by weight, between about 40% by weight and about 60% by weight, more than about 60% by weight, and the like. The sizes of the ice particles will be controlled, optionally by adding the components such as a water-soluble surfactant (e.g., hydrotropic molecule), a poloxamer (e.g., Pluronic™ F127 or P407) and/or lipids, to allow for flowability through a vessel of various sizes (e.g., needle gauge size of between about 7 and about 43). Vessels of various sizes are described in U.S. application Ser. No. 15/505,042 (Publication No. US2017/0274011), the disclosure relating to vessels for injection is incorporated by reference herein. Further, other methods may be used to condition the size of the ice particles to allow for flowability and/or injectability through a vessel of various sizes (e.g., using a filter or transferring the composition back and forth between two syringes). In some embodiments, the majority of ice particles have a diameter that is less than about half of the internal diameter of the lumen or vessel used for injection. For example, ice particles can be about 1.5 mm or less in diameter for use with a 3 mm catheter.

In some embodiments, one or more excipients may be included in the cold slurry. Excipients can constitute less than about 10% volume by volume (v/v), between about 10% v/v and about 20% v/v, between about 20% v/v and about 30% v/v, between about 30% v/v and 40% v/v, and greater than about 40% v/v of the cold slurry. Various added excipients can be used to alter the phase change temperature of the cold slurry (e.g., reduce the freezing point), alter the ice percentage of the cold slurry, alter the viscosity of the cold slurry, prevent agglomeration of the ice particles, prevent dendritic ice formation (i.e., crystals with multi-branching “tree-like” formations, such as those seen in snowflakes), keep ice particles separated, increase thermal conductivity of fluid phase, or improve the overall prophylactic, therapeutic, or aesthetic efficacy of the flowable and/or injectable cold slurry. In the compositions described herein, such excipients may include hyaluronic acid, a poloxamer, a polysorbate (or other water-soluble surfactants such as hydrotropic substances), non-water-soluble substances, lipids (including lipid particles), which all prevent agglomeration of the ice particles, prevent dendritic ice formation (i.e., crystals with multi-branching “tree-like” formations, such as those seen in snowflakes), or keep ice particles separated, such that the cold slurry is flowable and/or injectable when it is removed from a freezer.

One or more freezing point depressants can be added as excipients to sterile water to form a cold slurry with freezing points below 0° C. (e.g., about −10° C.). Depressing the freezing point of the cold slurry allows it to maintain flowability and remain injectable while still containing an effective percentage of ice particles. Suitable freezing point depressants include salts (e.g., sodium chloride, betadex sulfobutyl ether sodium), ions, Lactated Ringer's solution, sugars (e.g., glucose, sorbitol, mannitol, hetastarch, sucrose, (2-Hydroxypropyl)-β-cyclodextrin, or a combination thereof), biocompatible surfactants such as glycerol (also known as glycerin or glycerine), other polyols (e.g., polyvinyl alcohol, polyethylene glycol 300, polyethylene glycol 400, propylene glycol), other sugar alcohols, or urea, and the like. Other exemplary freezing point depressants are disclosed in U.S. application Ser. No. 15/505,042 (Publication No. US2017/0274011), the disclosure relating to slurry composition ingredients is incorporated in its entirety herein.

The present disclosure describes compositions that, when frozen, result in flowable and/or injectable cold slurries. In some embodiments, the compositions of the present disclosure do not require processing or manipulation to be flowable and/or injectable. However, manipulation may be used in other embodiments to further improve flowability and injectability or to promote consistency. In some embodiments, the compositions comprise a suspension of fluid with high water content (e.g., between about 70% and 80%, between about 80% and 90%, or greater than about 90%), a solute used to depress the freezing point (e.g., glycerol), and a hyaluronic acid. In some embodiments, to further improve flowability and injectability of the cold slurry, the solution may contain one or more of a lipid, a non-water-soluble compound, or a water-soluble surfactant such as a hydrotropic compound (e.g., a polysorbate) or a poloxamer (e.g., P407). In some embodiments, the solution further comprises an additional excipient, such as sodium chloride or sodium phosphate, to form, for example, saline or a phosphate-buffered saline.

The present disclosure provides for various compositions. In some embodiments, the composition contains an effective amount of hyaluronic acid to create a flowable and/or injectable cold slurry. Without intending to be bound by any particular theory, it is believed that the hyaluronic acid in the composition facilitates the formation of small ice crystals when the solution is exposed to freezing temperatures (e.g., between about −20° C. and −15° C., between about −15° C. and −10° C., between about −10 and −5° C., or in some embodiments about −10° C.). In some embodiments, a composition comprising hyaluronic acid is transformed into a flowable and/or injectable and cold slurry having ice particles when placed into a standard freezer without requiring the application of any mechanical agitation or additional treatment to the cold slurry.

In some embodiments, the composition further comprises an effective amount of a water-soluble surfactant such as a hydrotropic compound (e.g., a polysorbate), to create a flowable and/or injectable ice slurry. In some embodiments, the water-soluble surfactant is a poloxamer (or Pluronic™) molecule. In some embodiments, any surfactant with a hydrophilic-lipophilic balance (HLB) value greater than 10 is considered water-soluble. In some embodiments, the water-soluble surfactant in the composition is in a concentration of between about 0.01% (w/w) and 0.5% (w/w), between about 0.5% (w/w) and 1% (w/w), between about 1% (w/w), and 2% (w/w), between about 2% (w/w) and 5% (w/w), between about 5% (w/w) and 10% (w/w), or greater than about 10% (w/w). Without intending to be bound by any particular theory, it is believed that the water-soluble surfactant further serves to prevent ice particles from growing too large when the composition is exposed to freezing temperatures (e.g., about −5° C. or less); large ice particles may prevent the composition from being flowable or injectable.

In some embodiments, the composition further comprises an effective amount of one or more of a hyaluronic acid, an optional hydrotropic molecule, and a lipid, or non-water-soluble particles, to create a flowable and/or injectable cold slurry in the form of an emulsion. In some embodiments, an emulsion is any composition described herein that comprises a lipid. In some embodiments, the lipids in the composition are assembled into lipid particles having one or more morphologies known in the art (e.g., lamellar or non-lamellar structures, bilayer and non-bilayer structures, including liposomes, lipid nanoparticles, micelles, etc.). The lipid particle morphology of the present disclosure may be determined by any method known in the art such as by CryoTEM. In some embodiments, the lipid particles in the composition are between about 5 μm and about 300 μm in diameter. In some embodiments, the lipid particles are about 250 μm in diameter. In some embodiments, the lipid particles in the composition are between about 5 μm and 20 μm in diameter, or between about 8 μm and 14 μm in diameter. Without intending to be bound by any particular theory, it is believed that the lipids or non-water-soluble particles prevent ice particles from growing too large when the composition is exposed to freezing temperatures such that the composition is no longer flowable or injectable.

In some embodiments, the hyaluronic acid is of a natural or synthetic origin. In some embodiments, the hyaluronic acid has a molecular weight of between about 250 kDa and 5,000 kDa. In some embodiments, the hyaluronic acid has a molecular weight of about 1,000 kDa. In some embodiments the concentration of hyaluronic acid in the composition is between about 0.01% (w/w) and 2% (w/w). In some embodiments, the concentration of hyaluronic acid in the composition is between about 0.1% (w/w) and 1% (w/w). In some embodiments, the concentration of hyaluronic acid in the composition is between about 0.5% (w/w) and 1.5% (w/w). In some embodiments, the concentration of hyaluronic acid in the composition is between about 0.05% (w/w) and 0.75% (w/w). In some embodiments, the concentration of hyaluronic acid in the composition is about 0.5% (w/w). In some embodiments, the hyaluronic acid in the composition is in the form of a hyaluronic acid gel (i.e., having high viscosity).

In some embodiments, the excipient is selected from the group consisting of a salt, an ion, Lactated Ringer's solution, a sugar, a biocompatible surfactant, a polyol, and a combination thereof. In some embodiments, the excipient is a polyol. In some embodiments, the polyol is glycerol. In some embodiments, the glycerol concentration of the composition is between about 12% and 25% (w/w). In some embodiments, the glycerol concentration of the composition is about 20% (w/w).

In some embodiments, the composition includes a second excipient. In some embodiments, the second excipient is sodium chloride or sodium phosphate, to form, for example, saline or a phosphate-buffered saline.

In some embodiments, the composition includes a third excipient. In some embodiments, the third excipient is a water-soluble surfactant. In some embodiments, the third excipient is a hydrotropic compound. In some embodiments, the third excipient is a polysorbate.

In some embodiments, the composition includes a Pluronic™ (also referred to as “poloxamers”). In some embodiments, the poloxamer forms a macromolecular assembly, like a micelle. In some embodiments, the macromolecular assembly is a micelle (or a “poloxamer micelle”). In some embodiments, the Pluronic or the poloxamer is selected from the group consisting of Pluronic L31, Pluronic L35, Pluronic F38, Pluronic L43, Pluronic L44, Pluronic L61, Pluronic F68, Pluronic F77, Pluronic L81, Pluronic P84, Pluronic P85, Pluronic F77, Pluronic F87, Pluronic L92, Pluronic F98, Pluronic L101, Pluronic P103, Pluronic P104, Pluronic P105, Pluronic F108, Pluronic L121, Pluronic P123, Pluronic F127, poloxamer 101, poloxamer 105, poloxamer 108, poloxamer 122, poloxamer 123, poloxamer 124, poloxamer 181, poloxamer 183, poloxamer 188, poloxamer 212, poloxamer 215, poloxamer 217, poloxamer 231,poloxamer 234, poloxamer 235, poloxamer 237, poloxamer 238, poloxamer 282, poloxamer 284,poloxamer 288, poloxamer 331, poloxamer 333, poloxamer 334, poloxamer 335, poloxamer 338,poloxamer 401, poloxamer 402, poloxamer 403, poloxamer 407, poloxamer 105 benzoate, poloxamer 182 dibenzoate, and a combination thereof. In some embodiments, the Pluronic or poloxamer is Pluronic F127. In certain embodiments, the poloxamer is poloxamer 407.

In certain embodiments, the concentration of the poloxamer is between about 0% and 10% (w/w). In certain embodiments, the concentration of the poloxamer is between about 0.1% and 10% (w/w). In some embodiments, the concentration of the poloxamer is about 5% (w/w). In certain embodiments, the Pluronic™ is Pluronic™ F127, wherein the concentration of Pluronic™ F127 is about 5% (w/w). In some embodiments, the poloxamer is poloxamer 407, wherein the concentration of poloxomer 407 is about 5% (w/w).

In some embodiments, the composition includes a lipid. In some embodiments, the composition includes a plurality of lipids in the form of a liposome formed from phospholipids (e.g., soy PC). The lipid may be of any type (e.g., phospholipid, cholesterol, conjugated lipid, or a combination thereof) or the composition may include any other non-water-soluble substance instead of a lipid. The lipid (or lipid particle)or non-water-soluble substance is present in a relatively high concentration, preferably between about 6% (w/w) and 28% (w/w) of the composition. Without intending to be bound by any particular theory, it is believed that the lipids (or lipid particles such as liposomes) or non-water-soluble substances create an emulsion when the composition is exposed to freezing temperatures (between about −25° C. and −15° C., between about −15° C. and −10° C., between about −15° C. and −5° C., between about −10° C. and −5° C., or in some embodiments about −10° C.) because these substances prevent large crystalline formations of ice. This allows the composition to have ice particles while also being flowable and/or injectable. In some embodiments, the composition further includes a lipid particle. In some embodiments, the lipid particle is a liposome. In some embodiments, the lipid particle is a micelle. In some embodiments, the lipid particle is comprised of a phospholipid. In some embodiments, the phospholipid is selected from the group consisting of 1,2-distearoyl-sn-glycero-3-phosphocholine (DSPC), egg sphingomyelin (DPSM), dipalmitoylphosphatidylcholine (DPPC), dicethylphosphate (DCP), L-a-phosphatidylcholine (PC), phosphatidylethanolamine, (PE), phosphatidylserine (PS), phosphatidylglycerol (PG), L-a-phosphatidylcholine (soy PC), and a combination thereof. In some embodiments, the lipid is L-a-phosphatidylcholine (soy PC). In some embodiments, the lipid concentration in the composition is between about 0% and 30% (w/w).

In some embodiments, the composition further includes ethanol. In some embodiments, the concentration of ethanol in the composition is between about 0.01% and 0.1%. In some embodiments, the concentration of ethanol in the composition is about 0.07% or less.

In some embodiments, the composition comprises glycerol, hyaluronic acid, and Pluronic™ F127 in water, saline, or phosphate-buffered saline. In some embodiments, the composition comprises glycerol, hyaluronic acid, and Pluronic™ F127 in saline, wherein the hyaluronic acid has a molecular weight of about 1000 kDa. In some embodiments, the composition comprises about 20% (w/w) glycerol, about 0.5% (w/w) hyaluronic acid, and about 5% (w/w) Pluronic™ F127 in water, saline, or phosphate-buffered saline. In some embodiments, the composition comprises about 20% (w/w) glycerol, about 0.5% (w/w) hyaluronic acid, and about 5% (w/w) Pluronic™ F127 in water, saline, or phosphate-buffered saline, wherein the hyaluronic acid has a molecular weight of about 1000 kDa.

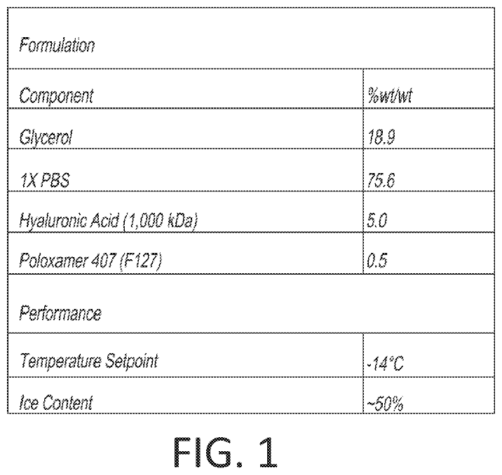

In some embodiments, the composition comprises glycerol, PBS, hyaluronic acid, and poloxamer 407 (Pluronic™ F127). In certain embodiments, the composition comprises glycerol having a concentration of approximately 18.9% (w/w), 1× PBS having a concentration of approximately 75.6% (w/w), hyaluronic acid having a molecular weight of 1000 kDa and a concentration of about 5% (w/w), and poloxamer 407 (Pluronic™ 127) having a concentration of about 0.5% (w/w). See, e.g., FIG. 1. In certain embodiments, the composition comprises a temperature setpoint of about −14° C. See id. In certain embodiments, the composition has an ice content wherein the ice content is about 50%. In certain embodiments, the composition is the composition shown in FIG. 1. In certain embodiments, the composition includes a relatively small percentage of hyaluronic acid, between about 0.5% (w/w) and 1.5% (w/w), wherein the molecular weight of the hyaluronic acid is about 1,000 kDa. In such embodiments, the composition also contains a concentration of glycerol of between about 15% (w/w) and 25% (w/w), and a small percentage of a polysorbate between about 0.25% (w/w) and 1.5% (w/w). This allows the composition to have ice particles while also being flowable and/or injectable once frozen.

In some embodiments, the composition is filled into a container with a volume less than 10 mL and with a shape that results in maximum surface area of the container walls. Without intending to be bound by any particular theory, it is believed that the large surface area to volume ratio facilitates an increased freezing rate to further prevent large ice crystal formation and therefore improve flowability and injectability. In some embodiments, a total injection volume of the cold slurry into a patient, which is optionally injected via multiple containers and multiple injections, is between about 5 mL and 10 mL, between about 10 mL and 20 mL, between about 20 mL and 30 mL, between about 30 mL and 40 mL, between about 40 mL and 50 mL, between about 50 mL and 60 mL, between about 60 mL and 70 mL, or more than about 70 mL. In some embodiments, the total injection volume is about 60 mL.

Methods of creating cold slurries by formulating a solution that prevents the formation of large ice crystals are described in PCT Application No. PCT/US20/43280 and the '494 PCT, incorporated by reference in its entirety herein. Described herein is an unexpected method of forming a cold slurry by creating a composition having hyaluronic acid, water, and at least one excipient, wherein the creation of the cold slurry does not require any mechanical manipulation or agitation of the composition. In some embodiments, the composition further includes a poloxamer. In some embodiments, the composition includes a plurality of lipids. In some embodiments, the injectability of the composition is improved by utilizing minor, mechanical agitation.

In some embodiments, the composition described herein is a homogenous mixture such that the composition media throughout the container is uniform and the components are distributed evenly. In some embodiments, the addition of a poloxamer, hyaluronic acid, and glycerol prevents the formation of large ice crystals such that a flowable cold slurry can be injected into a subject immediately after removal of the cold slurry from the freezer or another cold environment.

The compositions provided herein can be provided in a syringe or other container. The syringe or other container may also include a visible temperature indicator that can allow for visual monitoring of the temperature of the slurry, or the approximate temperature of the slurry. The temperature indicator can be a temperature sensing label, sticker, marker, crayon, lacquer, pellet, etc., including reversible temperature labels that can dynamically track temperature changes. The temperature indicator can be located inside the syringe or other container (e.g., a pellet placed directly into the internal solution), on the inside walls of the syringe or other container, on the outside walls of the syringe or other container, or in any location that allows for visual tracking of the temperature of the contents inside the syringe or other container. In certain embodiments, the composition is provided in a syringe and the syringe is placed in a freezer. The syringe is withdrawn from the freezer following a period of time (e.g., 24 hours) that is sufficient for ice crystals to form in the syringe. After removing the syringe from the freezer, the syringe contents are monitored, for example, using a temperature indicator (e.g., infrared sensor or external temperature-sensitive indicator). Once the syringe contents reach a predetermined temperature, the syringe is connected to a second syringe and subjected to BAF processing (e.g., three BAF cycles). The slurry is then injected to a patient or a subject. In some embodiments, alternative methods of processing the slurry can be used, for example, pushing the slurry through a filter; including a wire between the two syringe openings that the slurry has to be injected around; providing internal components in the syringe that provide such processing such as magnets, internal blades, or an internal formed wire (e.g., a spring). In some embodiments, the BAF processing or other processing occurs after the container's contents reach a predetermined temperature after being removed from a freezer. In some embodiments, the BAF processing or other processing occurs immediately after the container is withdrawn from the freezer, before it reaches a predetermined temperature. Without intending to be bound by any particular theory, it is believed that the further mechanical processing reduces ice crystal size in the cold slurry and makes the ice crystals easier to inject through a needle.

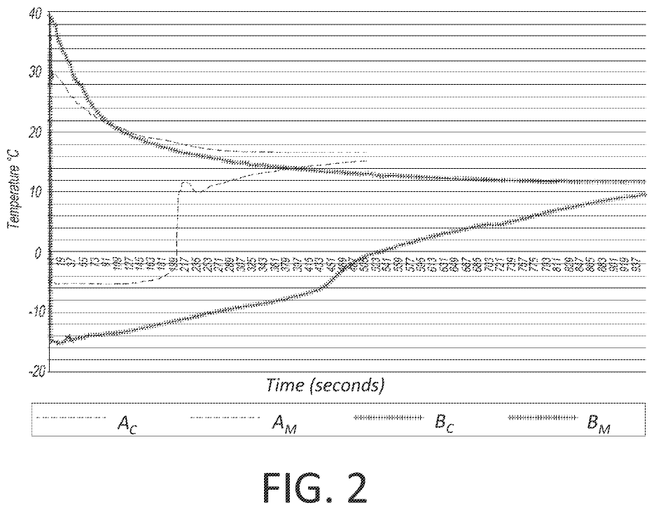

Referring to FIG. 2, two different cold slurry compositions (batches) are characterized with respect to their temperature profiles and ice content over time. The different cold slurry batches were placed into a copper plate that is heated to 40° C. and has thermocouple wires that measure changes in temperature of the cold slurry over time. The plotted data shows temperature change over time in seconds for two different cold slurry compositions: 1) containing 12.6% (w/w) glycerol and PBS (represented by traces A_{C} and A_{M}), and 2) containing 0.5% (w/w) soy-PC, 0.08% (w/w) EtOH, 0.75% (w/w) hyaluronic acid (1,000 kDa), 16% (w/w) glycerol, and PBS (represented by traces B_{C} and B_{M}). The temperatures are measured at two different positions for each cold slurry: one thermocouple is embedded inside of the copper plate (traces A_{C}, B_{C}) and the other thermocouple is located in the middle of the copper plate exposed to the outside of the plate (traces A_{M}, B_{M}). When a batch of cold slurry is first introduced into the copper plate, the thermocouple wire embedded inside the plate (traces A_{C}, B_{C}) initially measures the warm temperature of the heated plate (e.g., 31° C. for trace Ac at timepoint 0) and then reaches an equilibrium at a lower temperature due to the cooling effect of the introduced cold slurry (e.g., 20° C. for trace A_{C} at around 2 minutes). On the other hand, for the thermocouple wire located in the middle of the plate (traces A_{M}, B_{M}), when a cold slurry is first introduced into the copper plate it immediately contacts the thermocouple wire since that wire is exposed. This causes an initially negative temperature reading in the middle position due to the crystallized cold slurry contacting the wire (e.g., −4° C. for trace A_{M} at timepoint 0) followed by an equilibrium at a warmer temperature as the cold slurry begins to melt on the heated plate (e.g., 14° C. for trace A_{M} at around 6 minutes). The thermocouple wire exposed to the outside of the plate (traces A_{M}, B_{M}) can be used to detect phase transitions during which the crystallized cold slurry begins to melt. The graph shows that the slurry composition with hyaluronic acid (traces B_{C} and B_{M}) have a progressive phase transition. The graph also shows that the cold slurry batch having the hyaluronic acid composition (traces B_{C} and B_{M}) reaches equilibrium (as measured by the two thermocouple wire positions) in a similar time frame and at similar temperatures of between about 10° C. and 14° C. depending on the location of the thermocouple (inside/middle). On the other hand, the cold slurry with a different composition (lacking hyaluronic acid; traces A_{C} and A_{M}) has a different temperature profile from the composition comprising hyaluronic acid, reaching an equilibrium sooner at the temperature of between about 15° C. and 17° C. depending on the location of the thermocouple (inside/middle). FIG. 2 therefore demonstrates that cold slurries can have different compositions that are designed to have different temperature profiles or can be designed to perform equivalently.

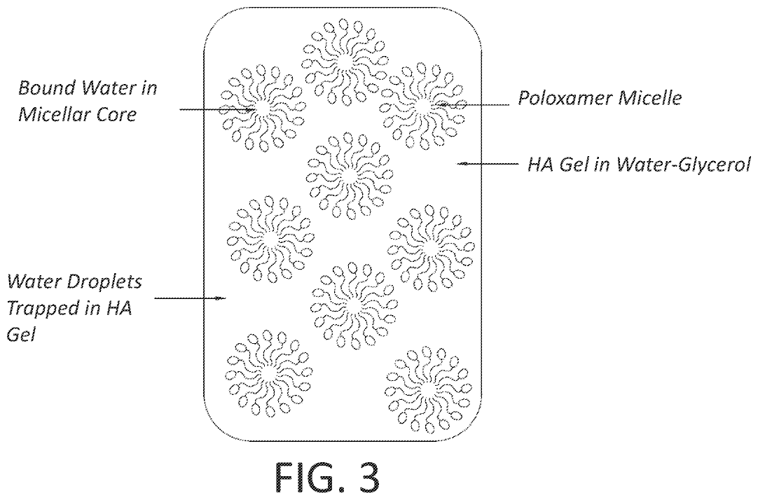

FIG. 3 depicts an embodiment of a composition comprising poloxamer micelles, glycerol, and a hyaluronic acid gel. In this embodiment, the composition includes micelles formed by a Pluronic™ (e.g., Pluronic™ F127) with an aqueous core (e.g., containing water). In some embodiments, the media external to the micelles contains hyaluronic acid gel in a water-glycerol and, optionally an ethanol solution (not shown). In some embodiments, water droplets are trapped within the external media (i.e., outside the micelles in the hyaluronic acid gel in a water-glycerol and, optionally an ethanol solution (not shown)). The embodiment of FIG. 3 is referred to herein as a “micellar dispersion” or a “micellar gel.” The micellar dispersion or micellar gel includes surfactant micelles that trap water where the micelles are suspended in the hyaluronic acid gel. In some embodiments, the morphology of the micelles in the composition are normal micelles (as depicted in FIG. 3) or reverse micelles. In some embodiments, lipid micelle compositions comprising hyaluronic acid are provided.

In some embodiments, an at least partially crystallized composition containing hyaluronic acid, glycerol, water, and saline (or PBS) contains sufficient ice particles to be flowable and/or injectable without the addition of other excipients upon being exposed to freezing temperatures (i.e., being placed in a freezer). In some embodiments, an at least partially crystallized composition containing hyaluronic acid, glycerol, and water contains sufficient ice particles to be flowable and/or injectable without the addition of other excipients upon being exposed to freezing temperatures (i.e., being placed in a freezer). In alternative embodiments, the addition of a poloxamer, a polysorbate, or a lipid further improves the flowability and injectability of the partially crystallized composition. It has also been discovered that increasing the rate of freezing of the material to a faster rate further improves flowability and injectability of the composition. Optimization of the freezing rate includes selecting a material for the container into which the composition is placed (e.g., a syringe), the geometry of the container, and the selection of the cold environment or freezer (e.g., the humidity of the freezer may be modulated to improve the flowability and/or injectability of the resulting cold slurry). It has also been found that the injectability or flowability of the partially crystallized composition can be improved by spacing the containers from one another when placed in a freezer.

In some embodiments, the composition (e.g., in the form of a liquid solution) may be packaged and sealed in a container such as a syringe. The syringe can be filled sterile (e.g., using aseptic procedures) or the syringe may be pre-filled, sealed, and then terminally sterilized (e.g., using autoclave or steam sterilization). The composition can also be provided in any other sealed container that can be terminally sterilized, such as a tube used for topical ointment, or a larger container used to then fill a plurality of syringes. In certain embodiments, the pre-filled syringe or other container can be terminally sterilized using gamma radiation, or autoclave or steam sterilization at a temperature of about 118° C. In certain embodiments, the pre-filled syringe or other container can be terminally sterilized using gamma radiation, or autoclave or steam sterilization at a temperature of between about 118° C. and 121° C. In a preferred embodiment, when using hyaluronic acid with a molecular weight of 1,000 kDa, the composition is terminally sterilized using steam because sterilization using radiation will affect the molecular weight of the hyaluronic acid. In certain embodiments, the composition is terminally sterilized using hyaluronic acid with a molecular weight of 1,000 kDa, the composition is terminally sterilized using a low dose radiation or terminally sterilized using radiation to irradiate a still-frozen cold slurry.

In some embodiments, the composition inside the syringe (or other container) is transformed into a flowable and/or injectable cold slurry by placing the pre-filled syringe (or other container) into a standard freezer, or other cold environment. In certain embodiments, a structure is provided to store one or more pre-filled syringes (or other containers) in a freezer or other cold environment, wherein the structure is configured to store a first pre-filled syringe (or container) of the one or more pre-filled syringes (or other containers) a pre-determined distance spaced apart from a second pre-filled syringe (or container) of the one or more pre-filled syringes (or other containers). In certain embodiments, a structure is provided to store a plurality of pre-filled syringes (or containers) a pre-determined distance spaced apart from one another to promote achieve even and rapid cooling of the cold slurry. See, e.g., Example 3. In certain embodiments, the pre-determined distance is at least 0.5 inches. In certain embodiments, the pre-determined distance is about 0.5 inches. In certain embodiments, the pre-determined distance is about 0.5 inches, about 0.75 inches, about 1 inch, about 1.25 inches, about 1.5 inches, about 1.75 inches, about 2 inches, about 2.25 inches, about 2.5 inches, about 2.75 inches, about 3 inches, about 3.25 inches, about 3.5 inches, about 3.75 inches, about 4 inches, or more than about 4 inches. In some embodiments, the structure is a rack, a tray, or a box. In some embodiments, the composition may be flash-frozen using liquid nitrogen or other liquid cooling methods to speed up the process.

After freezing, in some embodiments, the syringe or container can be removed from the freezer, cold environment, or other method of freezing, and the cold slurry can be immediately injected or applied, optionally by topical application, for therapeutic benefit. In some embodiments, slurry can be applied directly to tissue following invasive surgical methods. In some embodiments, the cold slurry can be injected directly from the syringe using a needle. In certain embodiments, the needle is a 16G needle, a 17G needle, an 18G needle, a 19G needle, a 20G needle, a 21G needle, a 22G needle, a 23G needle, or a 24G needle. The cold slurry can also be removed from the container for a topical application when the container is removed from the freezer, such as by squeezing the container to dispel the cold slurry onto a targeted treatment site. In some embodiments, the cold slurry is in a flowable or injectable form immediately after being removed from the freezer without any further mechanical manipulation.

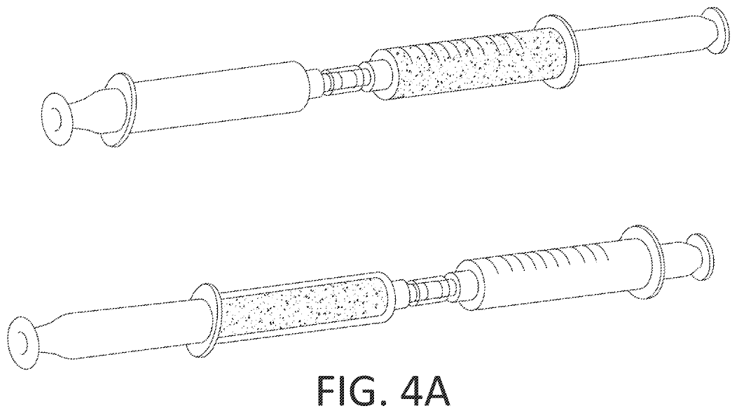

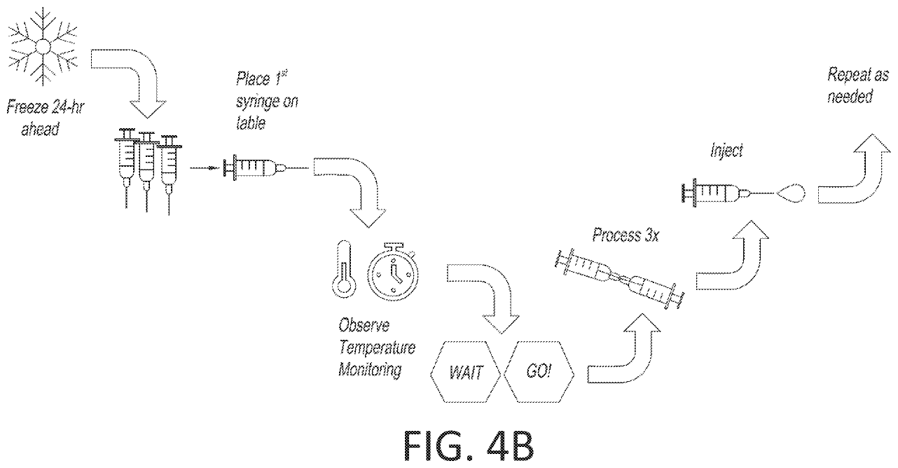

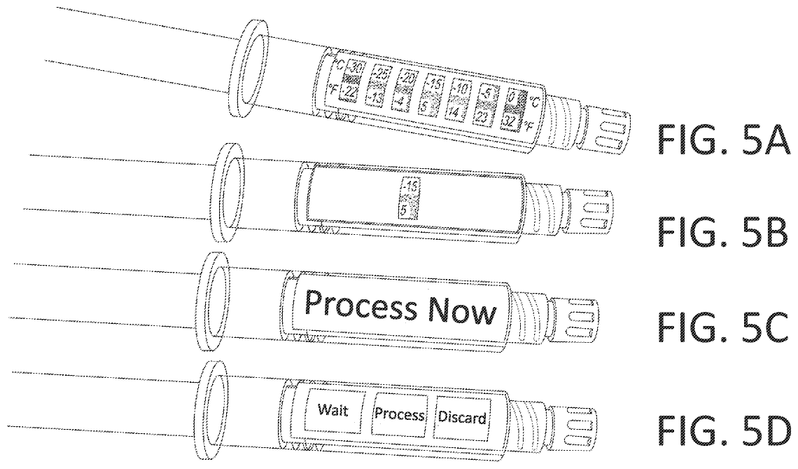

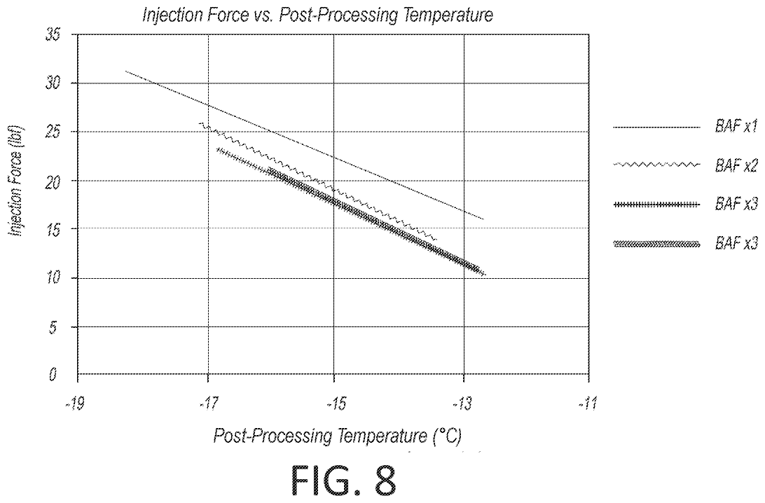

In certain embodiments, after being removed from the freezer, the cold slurry is subjected to mechanical manipulation to improve flowability or injectability prior to being injected or applied, optionally by topical application, for therapeutic benefit. In some embodiments, as shown in FIG. 4A, the cold slurry can be provided in a first syringe or container configured to be connected, optionally via a female Leur adapter, to a second syringe or container to facilitate “back-and-forth” (BAF) processing of the cold slurry. See, e.g., Example 5. In certain embodiments, as shown in FIG. 4A, a system is provided comprising a first sterile syringe or other container comprising a male Luer component and containing the cold slurry and a second sterile syringe or container comprising a male Luer component and containing no cold slurry, wherein the first syringe or container and the second syringe or container are connected by a sterile female Leur connector. In certain embodiments, the BAF processing constitutes: (1) pushing the cold slurry from the first syringe into the second syringe and (2) pushing the cold slurry from the second syringe back into the first syringe to complete one “back-and-forth” cycle. In certain embodiments, the cold slurry is subjected to one, two, three, four, or more BAF cycles. See Example 5; FIG. 8. In certain embodiments, the cold slurry is subjected to two BAF cycles. In certain embodiments, the cold slurry is subjected to three BAF cycles. An exemplary method comprising three back-and-forth cycles is shown in FIG. 4B. The exemplary method shown in FIG. 4B comprises freezing a first syringe for 24 hours, where the syringe holds the slurry composition, before removing the syringe from the freezer. Next, the temperature of the now cold slurry is observed using temperature monitoring (e.g., by using a temperature sensitive sticker as depicted in FIGS. 5A-5D) until the cold slurry reaches a pre-determined temperature. Once the pre-determined temperature is reached, the slurry is processed using a second syringe connected to the first syringe using a Leur connector and subjected to three BAF cycles. Following the three BAF cycles, the slurry is injected. This process may be repeated as necessary to prepare the number of syringes necessary for treatment. Pushing syringe contents from one syringe to another is a well-known technique that does not compromise the sterility of the syringe contents when sterilized components are used. Optionally, additional elements can be added to between the syringes to further break apart ice crystals. For example, one of the syringe openings could be covered with a filter or with a wire to break apart ice crystals. It is understood that the method shown in FIG. 4B can be used with the methods and systems described in International Publication No. WO 2022/055934, where a syringe containing the biocompatible composition described herein is transported to a point of care at ambient temperature, and is then placed into a freezer at the point of care to transform the composition into a cold slurry containing a plurality of ice crystals. The disclosure in the '934 PCT related to methods of transporting and transforming biocompatible compositions is incorporated by reference herein.

In some embodiments, after being removed from the freezer, the syringe is set aside and allowed to warm to a pre-determined temperature for injection or topical application. In certain embodiments, the pre-determined temperature is reached after the syringe contents undergo mechanical agitation. In some embodiments, the syringe or other container has an external temperature indicator configured to indicate when the pre-determined temperature is reached, e.g., by a color change or by displaying a message. In certain embodiments, the temperature indicator is a temperature-sensitive sticker or the like. The temperature indicator can allow for visual monitoring of the temperature of the contents of the syringe, or the approximate temperature of the contents of the syringe. The temperature indicator, e.g., temperature-sensitive sticker or the like, may display a range of temperatures, wherein a color change a region corresponding to in the pre-determined temperature range indicates that the pre-determined temperature has been reached. See, e.g., FIG. 5A. The temperature indicator can be a temperature sensing label, sticker, marker, crayon, lacquer, pellet, etc., including reversible temperature labels that can dynamically track temperature changes. The temperature indicator can be located inside the container (e.g., a pellet placed directly into the internal solution), on the inside walls of the container, on the outside walls of the container, or in any location that allows for visual tracking of the temperature of the contents inside the container. In certain embodiments, the pre-determined temperature is about −15° C. In certain embodiments, the pre-determined temperature is between about −19° C. and −11° C., between about −18° C. and −12° C., between about −17° C. and −13° C., or between about −16° C. and −14° C. In certain embodiments, a color change in a region corresponding to a colder temperature than the pre-determined temperature indicates that the cold slurry is colder than the pre-determined temperature to indicate that the cold slurry should be allowed to warm to the pre-determined temperature prior to injection or application. In certain embodiments, the colder temperature is about −20° C. In certain embodiments, the colder temperature is between about −20° C. and −25° C., between about −25° C. and −30 ° C., or below about −30° C. In certain embodiments, a color change in a region corresponding to a warmer temperature than the pre-determined temperature indicates that the cold slurry is warmer than advisable for injection or application to produce a therapeutic effect. In certain embodiments, the syringe may be refrozen if the warmer temperature is reached. For example, in certain embodiments, the warmer temperature is between about −10° C. and −8° C., between about −10° C. and −9° C., between about −9° C. and −8° C. between about −10° C. and −7° C., between about −10° C. and −6° C., between about −10° C. and −5° C., between about −10° C. and −4° C., between about −10° C. and −3° C., between about −10° C. and −2° C., between about −10° C. and −1° C., between about −10° C. and 0° C., −9° C. and 8° C., between about −9° C. and −7° C., between about- 9° C. and −6° C., between about −9° C. and −5° C., between about −9° C. and −4° C., between about −9° C. and −3° C., between about −9° C. and −2° C., between about −9° C. and −1 ° C., between about −9° C. and 0° C., between about −8° C. and −7° C., between about −8° C. and −6° C., between about −8° C. and −5° C. between about −8° C. and −4° C., between about −8° C. and −3° C., between about −8° C. and −2° C., between about −8° C. and −1° C., between about −8° C. and 0° C., or warmer than 0° C. In certain embodiments, the warmer temperature is between at least −10° C. and −8° C. or warmer. In certain embodiments, the warmer temperature is between at least −9° C. and −8° C. or warmer. In certain embodiments, the warmer temperature is between at least −10° C. and −9° C. or warmer. In certain embodiments, the indicator, e.g., temperature-sensitive sticker or the like, displays a temperature or range of temperatures indicating when the cold slurry has reached the pre-determined temperature. See, e.g., FIG. 5B. In certain embodiments, the indicator, e.g., temperature-sensitive sticker or the like, includes a region that displays a message indicating that the pre-determined temperature has been reached. See, e.g., FIG. 5C. For example, in certain embodiments, the indicator may display a message such as “Process Now,” “Process,” “Agitate,” “Go,” or any other suitable message. In certain embodiments, the indicator, e.g., temperature-sensitive sticker or the like, includes a region that displays a message indicating that the cold slurry is colder than the pre-determined temperature or warmer than the pre-determined temperature. See, e.g., FIG. 5D. For example, in certain embodiments, the indicator may display a message such as “Wait,” “Hold,” “Not Yet,” “Too Cold,” or any other suitable message to indicate that the cold slurry is colder than the pre-determined temperature. In certain embodiments, the indicator may display a message such as “Discard,” “Too Warm,” “Throw Away,” “Refreeze,” or any other suitable message to indicate that the cold slurry is warmer than the pre-determined temperature. In certain embodiments, a method is provided including temperature monitoring and back-and-forth mechanical agitation as depicted in FIG. 4B.

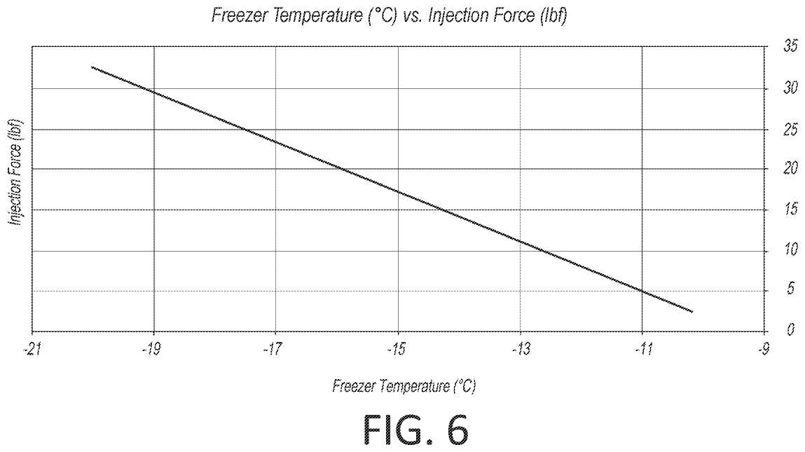

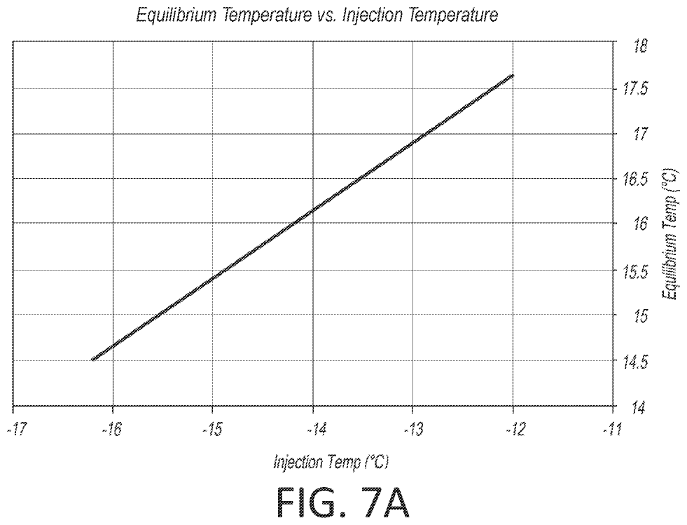

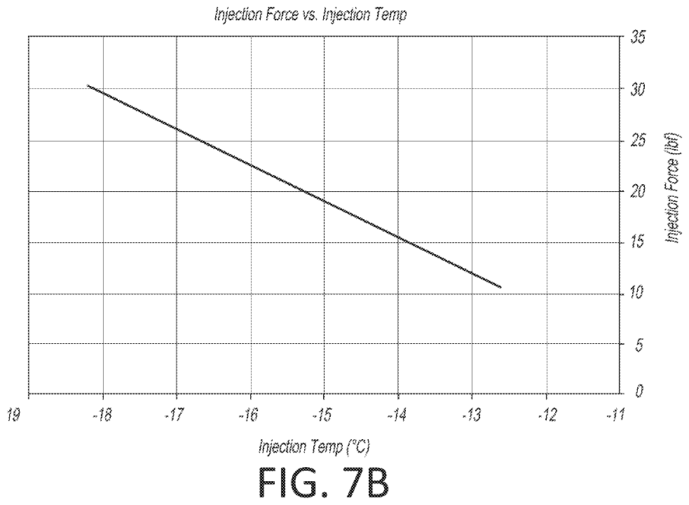

In one aspect, compositions are provided wherein the composition require an injection force of less than about 30 lbs plunger force to inject the composition through a 16G needle, a 17G needle, an 18G needle, a 19G needle, a 20G needle, a 21G needle, a 22G needle, a 23G needle, or a 24G needle syringe. In some embodiments, compositions provided herein require an injection force of less than about 30 lbs plunger force to inject the composition through a 17G or 18G needle. Compositions disclosed herein were prepared according to FIG. 2 and tested to examine whether there is a relationship between freezer temperature and injection force. As shown in FIG. 6, colder freezer temperatures require increased injection force to inject the cold slurry from a syringe because the composition in the syringe had more ice particles than at warmer temperatures. To determine ice content of a cold slurry, a test method and apparatus were designed that allow for ice content to be calculated based on final equilibrium temperature of the apparatus after a pre-determined amount of cold slurry is dispensed into the apparatus as described in Example 3. Referring to FIG. 7A, as equilibrium temperature increases, injection temperature increases. And as shown in FIG. 7B, as injection temperatures decrease, the injection force increased due to increased ice content. As the temperature of the cold slurry increases, the injection force is reduced. This result was observed for all syringe sizes (e.g., 3cc, 6cc) and needle gauges (e.g., 17G, 18G) tested. See, e.g., FIGS. 6, 7B, 8-9. Further, as temperature increases, it is possible to increase the overall reliability of injections, i.e., reduce the number of failed injections (e.g., injections where a spike in injection force is observed and slurry cannot be ejected from the syringe). By monitoring temperature of the cold slurry, it is possible to optimize injectability (reducing injection force) without reducing ice content below a therapeutic threshold for a given application. It has been found that the reliability of injection is greatly increased by following the steps of: (1) placing the syringe in a freezer to form a cold slurry containing a plurality of ice crystals; (2) waiting for the cold slurry to reach a pre-determined temperature after being withdrawn from the freezer; and (3) subjecting the cold slurry to three BAF cycles between two syringes. These results were consistently observed in injections≥5 mL (e.g., 6cc injections). In another aspect, mechanical agitation of the cold slurry according to methods described herein, e.g., by subjecting the cold slurry to one or more back-and-forth cycles, injection force is decreased. See, e.g., FIGS. 8, 10-11; Example 5.

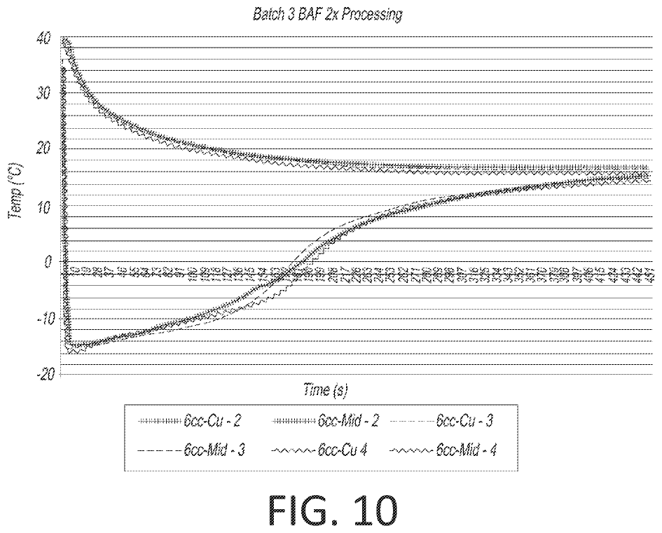

Referring to FIGS. 1, 6cc cold slurry compositions (batches) prepared according to the description below are characterized with respect to their temperature profiles and ice content. The different cold slurry batches were placed into a copper plate that is heated to 40° C. and has thermocouple wires that measure changes in temperature of the cold slurry over time. The plotted data shows temperature change over time for three batches of cold slurry compositions prepared according to FIG. 1 containing 18.9% (w/w) glycerol, 75.6% (w/w) 1×PBS, 5.0% (w/w) 1000 kDa hyaluronic acid, and 0.5% (w/w) poloxamer 407 (Pluronic™ F127). The three batches of cold slurry composition were then subjected to two BAF processing cycles as described herein. The temperatures were measured at two different positions for each cold slurry: embedded inside of the copper plate (traces A_{C}, B_{C}, C_{C}) and in the middle of the copper plate exposed to the outside of the plate (traces A_{M}, B_{M}, C_{M}). When a batch of cold slurry is first introduced into the copper plate, the thermocouple wire embedded inside the plate (traces A_{C}, B_{C}, C_{C}) initially measures the warm temperature of the heated plate (e.g., 35-40° C. for traces A_{C}, B_{C}, and C_{C} at timepoint 0) and then reaches an equilibrium at a lower temperature due to the cooling effect of the introduced cold slurry (e.g., about 20° C. for traces A_{C}, B_{C}, and C_{C} at around 2 minutes). On the other hand, for the thermocouple wire located in the middle of the plate (traces A_{M}, B_{M}, C_{M}), when a cold slurry is first introduced into the copper plate it immediately contacts the thermocouple wire since that wire is exposed. This causes an initially negative temperature reading in the middle position due to the crystallized cold slurry contacting the wire (e.g., −4° C. for trace A_{M} at timepoint 0) followed by an equilibrium at a warmer temperature as the cold slurry begins to melt on the heated plate (e.g., between about 12° C. and 14° C. for traces A_{M}, B_{M}, and C_{M} at around 6 minutes). The thermocouple wire exposed to the outside of the plate (traces A_{M}, B_{M}, C_{M}) can be used to detect phase transitions during which the crystallized cold slurry begins to melt. The graph shows that all cold slurry compositions tested (each prepared as described in FIG. 1 and subjected to two BAF cycles) have a progressive phase transition. The graph also shows that the cold slurry batches prepared in this way consistently reach equilibrium (traces A_{M}, B_{M}, and C_{M} as measured by the two thermocouple wire positions) in a similar timeframe and at similar temperatures. FIG. 10 therefore demonstrates that cold slurries designed according to the present disclosure produce consistent temperature profiles and ice content when subjected to mechanical agitation.

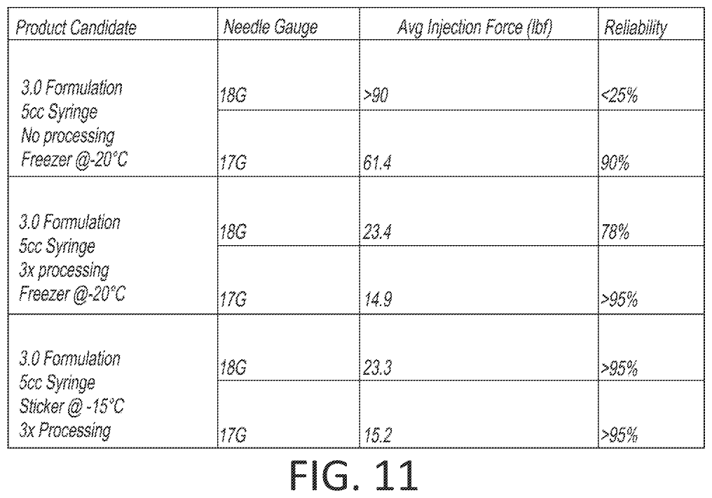

With reference to FIG. 11, characterizations of slurry compositions prepared to FIG. 1 and subjected to differing processing techniques and injected using an 18G or a 17G needle are provided. For all tests, cold slurries were held in a 5cc syringe. Reliability is characterized by quantifying the percentage of injections that did not exhibit an injection force spike above a predetermined limit. For the results presented in FIG. 11, the predetermined limit is 40 lbf. First, injection force was measured for cold slurries frozen at −20° C. and not subjected to further processing. Injection reliability was less than 20% using an 18G needle and 90% using a 17G needle, meaning the cold slurry could be injected through an 18G needle less than 20% of the time and through a 17G needle 90% of the time. Second, injection force was measured for cold slurries frozen at −20° C. and subjected to three BAF cycles after removal from the freezer but without waiting and temperature monitoring. Injection reliability was 78% using an 18G needle and greater than 95% using a 17G needle. Finally, injection force was measured for cold slurries frozen at −20° C., allowed to warm to −15° C. as determined by temperature monitoring, and subjected to three BAF cycles. Injection reliability was greater than 95% using an 18G needle and a 17G needle. Accordingly, FIG. 11 demonstrates that greater than 95% injection reliability can be achieved when slurry is subjected to three BAF cycles after being frozen at −20° C. and injected using a 17G needle. FIG. 11 further demonstrates that greater than 95% reliability can be achieved using both an 18G needle and a 17G needle when slurry is subjected to three BAF cycles after being frozen at −20° C. and allowed to warm to −15° C. as determined by temperature monitoring.

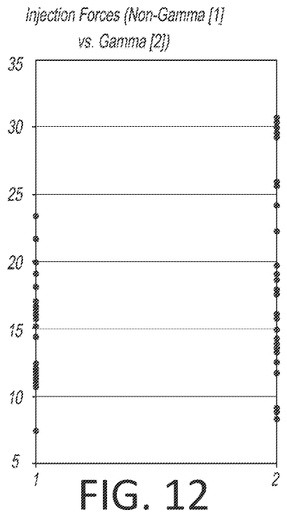

Referring to FIG. 12, results are presented from experiments testing the effect of syringes containing slurry composition were sterilized using radiation (gamma/e-beam sterilization). Results demonstrated that radiation sterilization using gamma/e-beam sterilization resulted in an increase in the injection force required to eject the cold slurry from the syringe. See FIG. 12. Without wishing to be bound to a particular theory, this increase in injection force required may be due to an effect on the molecular weight of hyaluronic acid in the composition. In some experiments, syringes containing slurry composition were sterilized using autoclave sterilization (steam sterilization) at varying temperatures (e.g., at about 118° C. or at about 121° C.). It was observed that, unlike radiation sterilization, autoclave or steam sterilization did not increase injection force required to eject cold slurry from the syringe.

The compositions described herein can be used for a variety of applications. After a composition in accordance with some embodiments of the present disclosure has been exposed to freezing temperatures such that it forms a flowable cold slurry, it can be administered topically to an area for therapeutic treatment. Methods of topical administration of cold slurries to the ocular surface are described in International Patent Application No. PCT/US21/24514, the disclosure related to therapeutic use of the slurry is incorporated by reference in its entirety herein. The compositions described herein can also be used to form a flowable and/or injectable cold slurry that can be injected into the targeted treatment area for therapeutic effect. Injection methods for cold slurries as described in International Patent Application No. US2017/0274078, the disclosure related to therapeutic use of injected slurry is incorporated by reference in its entirety herein.

The devices, systems, compositions, and methods disclosed herein are not to be limited in scope to the specific embodiments described herein. Indeed, various modifications of the devices, systems, and methods in addition to those described will become apparent to those of skill in the art from the foregoing description.

### EXAMPLES

### Example 1—Methods for Preparing a Cold Slurry Composition Comprising Hyaluronic Acid and a Poloxamer

Cold slurry compositions were prepared and tested by varying the following components: glycerol, hyaluronic acid, lipids, and a poloxamer (e.g., Pluronic™ F127). Compositions were prepared with glycerol content ranging from about 12-25% w/w. Compositions further included hyaluronic acid content ranging from about 0.1-1% w/w of hyaluronic acid having a molecular weight ranging from 250-5000 kDa. Lipid content in said compositions ranged from 0-30% w/w. Poloxamer content (e.g., Pluronic™ F127) in said compositions ranged from between 0-30% w/w.

Formulations for cold slurry compositions were evaluated using two primary test methods. First, compositions were tested to measure injection force through various needle gauges (e.g., 17G or 18G). Using a force test stand, force was applied to a plunger of a syringe at a pre-determined speed and peak force required to express all material through a needle (e.g., a 17G or 18G needle). Second, ice content was characterized for the cold slurry compositions using an apparatus to test thermal capacity of the cold slurry compositions. Following evaluation, a formulation comprising 20% w/w glycerol, 0.5% w/w 1000 kDa hyaluronic acid, and 5% w/w Pluronic™ F127 was prepared in saline. See FIG. 1.

### Example 2—Evaluation of Cold Slurry Cooling Techniques and Freezer Spacing

Cold slurry compositions were prepared as described in Example 2. To optimize performance, different preparation methods were tested to determine the effect of the different preparation methods on injection force as described below. In this Example, experiments were conducted to determine whether freezing temperature affected injection force required to eject the cold slurry composition from a syringe. Results demonstrated that lower freezer temperatures required greater injection forces. See FIG. 6. Moreover, flash freezing cold slurry compositions at a temperature of about −60° C. required an increased injection force as compared to controls. In certain experiments, cold slurry in a plurality of syringes were placed in a freezer by spacing the syringes apart to achieve even and rapid cooling of the cold slurry. It was observed that if the syringes were placed too close together, freezing was adversely affected, and larger ice crystals were observed. By contrast, decreased injection force was observed when injecting cold slurry from syringes that were spaced farther apart in the freezer.

### Example 3—Injectable Frozen Slurry Temperature Monitoring for Performance Optimization at Point of Care

Lower injection forces are generally preferable for injecting cold slurries according to the present disclosure, however, it is desirable that the cold slurries are within a correct temperature range and not too warm to produce a therapeutic effect. It is desirable that the cold slurry maintain sufficient ice content because the ice content of the cold slurry allows the cold slurry withdraw energy from a target tissue and provide a therapeutic effect. If the ice content of the cold slurry is too low, the therapeutic effect may be diminished. Conversely, if the ice content of the cold slurry is too high, the cold slurry may not be injectable. In the present Example, experiments were conducted to determine the ice content of a slurry using a copper plate. The ice content was calculated based on a final equilibrium temperature of the apparatus after a pre-determined volume of slurry was dispensed into the apparatus. As shown in FIG. 7A, the relationship between injection temperature and the final equilibrium temperature are inversely related such that as the injection temperature increases, the ice content of the cold slurry is decreased. See FIG. 7A.

Further experiments demonstrated that as the temperature of the cold slurry increases, the injection force decreases. See FIG. 7B. Lower ice content also resulted in more reliable injections, i.e., the number of failed injections was reduced when the cold slurry had a lower ice content. However, as noted above, it is desirable that the cold slurry maintain sufficient ice content and temperature to maintain a therapeutic effect. Results demonstrated that, by targeting a pre-determined injection temperature, it was possible to optimize injection performance by controlling ice content. Accordingly, temperature monitoring apparatuses and systems were designed comprising an indicator that provides an indication of when the cold slurry has reached a pre-determined temperature, wherein the pre-determined temperature is selected to reduce the injection force required to eject the cold slurry from the syringe without reducing the ice content of the cold slurry below a therapeutic temperature. See, e.g., FIGS. 5A-D; see also FIG. 4B. Moreover, further temperature monitoring apparatuses and systems were designed comprising a plurality of indicators, wherein the plurality of indicators provides an indication of when the cold slurry is at a temperature that is too cold for injection; when the cold slurry has reached a pre-determined temperature, wherein the pre-determined temperature is selected to reduce the injection force required to eject the cold slurry from the syringe without reducing the ice content of the cold slurry below a therapeutic temperature; or when the cold slurry has reached a temperature that is too warm to achieve a desired therapeutic effect. See, e.g., FIGS. 5A, 5B-C, see also FIG. 4B.

### Example 4—Method for Processing a Cold Slurry Between a Plurality of Syringes to Improve Injectability

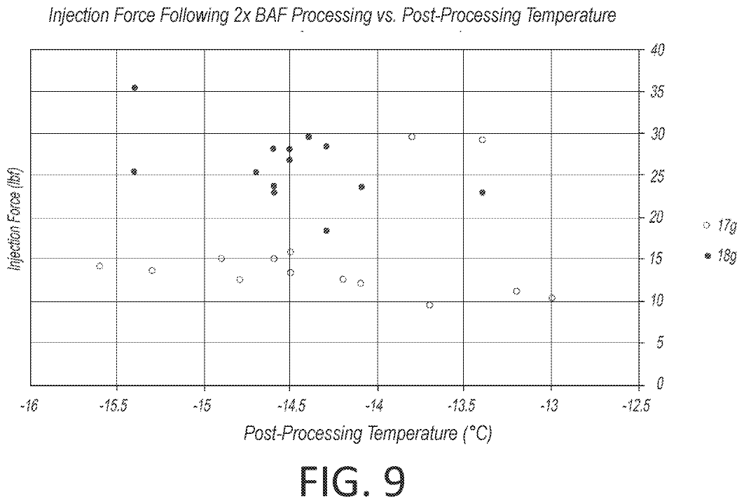

In certain experiments, different mechanical processing methods were tested. In some experiments, the cold slurry composition was prepared by connecting a first syringe and a second syringe using a connector, e.g., a Luer connector, and pushing the contents of the first syringe into the second syringe “back and forth.” See FIGS. 4A-B, 8. For example, in an experiment, a first syringe comprising a male Luer component and containing a cold slurry composition was connected to a second syringe comprising a male Luer component and not containing a cold slurry composition using a female-to-female Luer connector to connect the first syringe and the second syringe. See FIGS. 4A-B. Slurry was past back and forth between the first syringe and the second syringe, wherein one back and forth cycle included transferring the cold slurry from the first syringe to the second syringe followed by transfer back from the second syringe to the first syringe. It was observed that processing slurry by pushing the slurry back and forth significantly improved performance in injection tests and decreased the injection force required to eject the cold slurry from the syringe. See FIGS. 8-11. Without wishing to be bound by a particular theory, it is believed that performing one or more back and forth cycles decreases injection force by reducing the size of ice crystals in the cold slurry, dispersing ice crystals more evenly within the cold slurry, and/or increasing the temperature of the cold slurry by imparting shear forces to the cold slurry composition. Experiments were conducted to compare the injection force required to eject the slurry from the syringe following one, two, three, or four back and forth cycles were conducted. FIG. 8. Experiments demonstrated a significant reduction of injection force required to inject a cold slurry through a 6 ml syringe with a 17G needle attached. Id. Control cold slurries that were not subject to a back-and-forth cycle required injection forces of about 50-95 lbf. See, e.g., FIG. 11. Cold slurries subjected to a single back-and-forth cycle required a reduced injection force in the range of about 6-38 lbf. Id., see also FIGS. 8, 9. While experiments demonstrated that the injection temperature of the cold slurry is inversely correlated to the injection force, see FIG. 7B, performing one or more back-and-forth cycles further reduces the required injection force when the data was normalized relative to temperature. See FIG. 8. Results demonstrated that increasing the number of successive back-and-forth cycles to two cycles or three cycles further reduced the injection force. Id. It was observed that after three back and forth cycles, an additional decrease in injection force was not observed in the slurry subjected to a fourth back-and-forth cycle. Id. Additional experiments demonstrated that cold slurries subjected to two back-and-forth cycles were consistently injectable using less than about 30 lbf injection pressure over a range of post-processing temperatures using 17G and 18G needles. See FIG. 9.

Further experiments included a processing element in a connector, e.g., a Luer connector, between a first syringe and a second syringe pushing the contents of the first syringe into the second syringe back and forth as described above. It was observed that including a processing element in the connector produced a neutral result on injection force required.

### Example 5—Sterilization Methods for Cold Slurry Compositions Comprising Hyaluronic Acid

In certain experiments, various sterilization methods were examined. In some experiments, syringes containing slurry composition were sterilized using radiation (gamma/e-beam sterilization). Results demonstrated that radiation sterilization using gamma/e-beam sterilization resulted in an increase in the injection force required to eject the slurry from the syringe. See FIG. 12. Without wishing to be bound to a particular theory, this increase in injection force required may be due to an effect on the molecular weight of hyaluronic acid in the composition. In some experiments, syringes containing slurry composition were sterilized using autoclave sterilization (steam sterilization) at varying temperatures (e.g., at about 118° C. or at about 121° C.). It was observed that, unlike radiation sterilization, autoclave or steam sterilization did not increase injection force required to eject slurry from the syringe.

### Example 6—Method of Creating a Cold Slurry Composition Comprising Hyaluronic Acid and Soy PC

### Procedure to Make 0.5% Soy PC/HA/PBS/glycerol or 0.5% Tween 80/HA/PBS/glycerol for Ejection Test

1. Prepare PBS/Glycerol solution

Weigh 20 g glycerol in a 125 mL narrow mouth Erlenmeyer flask with a magnetic stir bar, added 80 mL 1×PBS, seal the flask's mouth with parafilm to prevent solvent evaporate. Stir the liquid at 500 rpm for 4 hours to obtain PBS/glycerol (41 by wt/wt, or 5/1 by vol/vol).
2. Prepare 0.75% HA/PBS/Glycerol solution

Weigh certain amount of HA 1000 kD (0.15 g to 0.75 g to make 20 g to 100 g of solution) in a 125 mL narrow mouth Erlenmeyer flask with a magnetic stir bar (8 mm×35 mm), add certain amount of PBS/Glycerol solution (19.85 g to 99.25 g to make 20 g to 100 g of solution). Seal the flask's mouth with parafilm to prevent solvent evaporate. Stir the liquid at 1200 rpm for 12-24 hours to obtain 0.75% HA/PBS/glycerol.
3. Dissolve soy PC in ethanol

Weigh 1 g of Soy PC in a 5 mL glass vial, add 0.2 mL ethanol by pipette, vortex and then incubate it at 45° C. for 12-24 h to make it a clear solution with light yellow color.
4. Prepare 0.5% Soy PC/HA/PBS/glycerol
   Example to make 20 g sample

Weigh 0.116 g above prepared soy PC in ethanol into a 125 mL narrow mouth Erlenmeyer flask with a disposable transfer pipette. Add 19.884 g 0.75% HA/PBS/glycerol. Put a magnetic stir bar (8 mm×35 mm) and seal the flask's mouth with parafilm to prevent solvent evaporate. Stir the liquid at 1200 rpm for 4-24 hours to obtain 0.5% Soy PC/HA/PBS/glycerol.
5. Prepare 0.5% Tween 80/HA/PBS/glycerol
   Example to make 20 g sample

Weigh 0.1 g Tween 80 into a 125 mL narrow mouth Erlenmeyer flask with a disposable transfer pipette. Add 19.9 g 0.75% HA/PBS/glycerol. Put a magnetic stir bar (8 mm×35 mm) and seal the flask's mouth with parafilm to prevent solvent evaporate. Stir the liquid at 1200 rpm for 4-24 hours to obtain 0.5% Tween 80/HA/PBS/glycerol.
6. Transfer the above prepared 0.5% Soy PC/HA/PBS/glycerol or 0.5% Tween

80/HA/PBS/glycerol into a 20 mL glass vial and sonicate 10 m. Then load the obtained liquid into 3×3 mL syringes by a 21G needle. Put the syringes into a freezer at −20° C.

After 12-24 h, take the syringes at −20° C. out of freezer and connect with a 18G needle for ejection test. The test should be performed within 1 minute of removal out of freezer.

What is claimed is:

1. A composition comprising:
an amount of water;
a hyaluronic acid; and
a first excipient, wherein the composition is configured to be formed into a flowable cold slurry comprising a plurality of ice crystals when the composition is exposed to a temperature of 0° C or less.

2. The composition of claim 1, wherein the composition further comprises a water-soluble surfactant.

3. The composition of claim 2, wherein the water-soluble surfactant is a poloxamer molecule.

4. The composition of claim 3, wherein the composition comprises a plurality of poloxamer molecules.

5. The composition of claim 3, wherein the composition comprises a poloxamer particle, and wherein the poloxamer particle comprises a plurality of poloxamer molecules.

6. The composition of claim 5, wherein the poloxamer particle is a micelle.

7. The composition of claim 3, wherein the poloxamer molecule is selected from the group consisting of poloxamer 101, poloxamer 105, poloxamer 108, poloxamer 122, poloxamer 123, poloxamer 124, poloxamer 181, poloxamer 183, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, poloxamer 182 dibenzoate, and a combination thereof.

8. The composition of claim 7, wherein the poloxamer is poloxamer 407, and wherein the concentration of the poloxamer 407 is between about 0.1% (w/w) and 10% (w/w).

9. The composition of claim 8, wherein the concentration of the poloxamer 407 in the composition is about 0.5% (w/w).

10. The composition of claim 1, wherein the first excipient is selected from the group consisting of a salt, an ion, Lactated Ringer's solution, a sugar, a biocompatible surfactant, a polyol, and a combination thereof.

11. The composition of claim 1, wherein the first excipient is glycerol.

12. The composition of claim 1, wherein a concentration of the glycerol in the composition is between about 12% and 25% (w/w).

13. The composition of claim 12, wherein the concentration of the glycerol in the composition is about 19% (w/w).

14. The composition of claim 1, wherein the composition further comprises a second excipient.

15. The composition of claim 14, wherein the second excipient is sodium chloride or sodium phosphate to form saline or phosphate-buffered saline.

16. The composition of claim 14, wherein the composition further comprises a third excipient.

17. The composition of claim 16, wherein the third excipient is a non-water-soluble substance.

18. The composition of claim 17, wherein the non-water-soluble substance is a lipid.

19. The composition of claim 18, wherein the lipid is selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-a-phosphatidylcholine (soy PC), phosphatidylethanolamine, (PE), phosphatidylserine (PS), phosphatidylglycerol (PG).

20. The composition of claim 1, wherein the composition is configured to form the plurality of ice crystals when the composition is exposed to a temperature of between about −25° C. and about −5° C.

21. The composition of claim 1, wherein the composition is configured to have an injection force of less than about 30 lbf when injected through a 16G needle, a 17G needle, an 18G needle, a 19G needle, a 20G needle, a 22G needle, a 23G needle, or a 24G needle.

22. The composition of claim 1, wherein the composition is configured to have an injection force of less than about 30 lbf when injected through a 17G needle or an 18G needle.

23. A method of preparing a cold slurry for administration to a patient at a clinical point of care, the method comprising:
preparing a composition comprising a hyaluronic acid and an amount of water;
adding a first excipient to the composition, wherein the excipient comprises a water-soluble surfactant; and
wherein the composition is configured to form a cold slurry comprising a plurality of ice particles when the composition is cooled to a temperature below about 0° C.

24. The method of claim 23, wherein the water-soluble surfactant is a hydrotropic molecule.

25. The method of claim 24, wherein the hydrotropic molecule is a poloxamer molecule.

26. The method of claim 25, wherein the poloxamer molecule is selected from the group consisting of poloxamer 101, poloxamer 105, poloxamer 108, poloxamer 122, poloxamer 123, poloxamer 124, poloxamer 181, poloxamer 183, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, poloxamer 182 dibenzoate, and a combination thereof.

27. The method of claim 25, wherein the poloxamer is poloxamer 407, and wherein the concentration of the poloxamer 407 is between about 0.1% (w/w) and 10% (w/w).

28. The method of claim 25, wherein the concentration of the poloxamer 407 in the composition is about 0.5% (w/w).

29. The method of claim 23, further comprising adding a second excipient to the composition, wherein the composition including the second excipient is configured to form the cold slurry when the composition is cooled to a temperature below about 0° C.

30. The method of claim 29, wherein the second excipient is selected from the group consisting of a salt, an ion, Lactated Ringer's solution, a sugar, a biocompatible surfactant, a polyol, and a combination thereof.

31. The method of claim 30, wherein the second excipient is glycerol.

32. The method of claim 31, wherein a concentration of the glycerol in the composition is between about 12 % and 25 % (w/w).

33. The method of claim 31, wherein the concentration of the glycerol in the composition is about 19 % (w/w).

34. The method of claim 31, further comprising adding a third excipient to the composition, wherein the composition including the second excipient and the third excipient is configured to form the cold slurry when the composition is cooled to a temperature below about 0° C.

35. The method of claim 34, wherein the third excipient is sodium chloride or sodium phosphate, to form saline or a phosphate-buffered saline.

36. The method of claim 34, further comprising adding a fourth excipient to the composition, wherein the composition including the second excipient, the third excipient, and the fourth excipient is configured to form the cold slurry when the composition is cooled to a temperature below about 0° C.

37. The method of claim 36, wherein the fourth excipient is a non-water-soluble substance.

38. The method of claim 37, wherein the non-water-soluble substance is a lipid.

39. The method of claim 38, wherein the lipid is selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-a-phosphatidylcholine (soy PC), phosphatidylethanolamine, (PE), phosphatidylserine (PS), phosphatidylglycerol (PG).

40. The method of claim 23, wherein the composition is configured to form the plurality of ice crystals when the composition is exposed to a temperature of between about −25° C. and about −5° C.

41. The method of claim 23, wherein the composition is configured to have an injection force of less than about 30 lbf when injected through a 16G needle, a 17G needle, an 18G needle, a 19G needle, a 20G needle, a 22G needle, a 23G needle, or a 24G needle.

42. The method of claim 23, wherein the composition is configured to have an injection force of less than about 30 lbf when injected through a 17G needle or an 18G needle.

43. A method of preparing a cold slurry for administration to a patient at a clinical point of care, the method comprising:
receiving a composition comprising a freezing point depressant and a hyaluronic acid; and
cooling the composition to a temperature below about 0° C. to form a cold slurry, wherein the cold slurry comprises a plurality of ice particles.

44. The method of claim 43, wherein the freezing point depressant is glycerol.

45. The method of claim 43, wherein the composition further comprises an amount of a poloxamer molecule.

46. The method of claim 45, wherein the poloxamer molecule is selected from the group consisting of poloxamer 101, poloxamer 105, poloxamer 108, poloxamer 122, poloxamer 123, poloxamer 124, poloxamer 181, poloxamer 183, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, poloxamer 182 dibenzoate, and a combination thereof.

47. The method of claim 46, wherein the poloxamer is poloxamer 407, and wherein the concentration of the poloxamer 407 is between about 0.1% (w/w) and 10% (w/w).

48. The method of claim 46, wherein the concentration of the poloxamer 407 in the composition is about 0.5% (w/w).

49. The method of claim 43, wherein the receiving a composition comprises receiving the composition within a container.

50. The method of claim 49, wherein the container is a first syringe.

51. The method of claim 50, further comprising connecting the first syringe to a second syringe, and processing the cold slurry through a back-and-forth cycle, wherein the back-and-forth cycle comprises pushing the cold slurry from the first syringe into the second syringe and pushing the cold slurry from the second syringe into the first syringe.

52. The method of claim 51, further comprising processing the cold slurry through a second, a third, or a fourth back-and-forth cycle.

53. The method of claim 49, wherein the container is a container configured for topical application.

54. The method of claim 53, wherein the container configured for topical application is a first tube.

55. The method of claim 54, further comprising connecting the first tube to a second tube and processing the slurry through a back-and-forth cycle, wherein the back-and-forth cycle comprises pushing the cold slurry from the first tube into the second tube and pushing the cold slurry from the second tube into the first tube.

56. The method of any one of claims 43-55, further comprising monitoring a temperature of the cold slurry.

57. The method of claim 56, wherein the monitoring comprises viewing a temperature sensitive indicator on a syringe or a container holding the cold slurry, wherein the temperature sensitive indicator is configured to indicate the temperature of the cold slurry.

58. The method of claim 57, wherein the temperature sensitive indicator is a temperature sensitive sticker.

59. The method of claim 57, wherein the temperature sensitive indicator provides a visual indication when the cold slurry reaches a pre-determined temperature.

60. The method of claim 59, wherein the pre-determined temperature is about −15° C.

61. The method of claim 59, wherein the pre-determined temperature is between about −19° C. and −11° C., between about −18° C. and −12° C., between about −17° C. and −13° C., or between about −16° C. and −14° C.

62. The method of any one of claims 59-61, wherein the temperature sensitive indicator is configured to provide a visual indication when the cold slurry is at a colder temperature than the pre-determined temperature.

63. The method of any one of claims 59-62, wherein the temperature sensitive indicator is configured to provide a visual indication when the cold slurry is warmer than the pre-determined temperature.

64. The method of claim 56, wherein the monitoring comprises viewing a thermometer.

65. The method of claim 56, wherein the monitoring comprises viewing a temperature monitoring component that is embedded within a container holding the cold slurry.

66. The method of claim 65, wherein the temperature component is provided in the container or is provided along a fluid path.

67. The method of any one of claims 56-66, wherein the monitoring further comprises listening for an audio indicator configured to indicate when the composition has reached a pre-determined temperature.

68. The method of any one of claims 56-67, wherein the composition is terminally sterilized.

69. The method of claim 68, wherein the composition is terminally sterilized via autoclave or steam sterilization.

70. The method of claim 69, wherein the autoclave or steam sterilization comprises subjecting the composition to a temperature between about 118° C. and 121° C.

71. The method of claim 70, wherein the temperature is about 118° C.

72. A cold slurry delivery system comprising:
a container holding a slurry composition, the container comprising a sterile barrier and a temperature indicator, wherein the container is configured to allow manual agitation of the slurry composition without breaking the sterile barrier.

73. The system of claim 72, wherein the container is a syringe or a tube.

74. The system of claim 73, wherein the container is configured to be connected to a second container.

75. The system of claim 74, wherein the container and the second container are configured to be connected using a connector.

76. The system of claim 75, wherein the container and the second container comprise a first syringe and a second syringe, wherein the first syringe and the second syringe each comprise a male Luer component.

77. The system of claim 76, wherein the connector comprises a female Luer component.

78. The system of claim 77, wherein the first syringe and the second syringe are connected using the female Luer component, and wherein the slurry composition is capable of being moved from the first syringe to the second syringe to manually agitate the slurry composition.

79. The system of any one of claims 72-78, further comprising a temperature sensitive indicator provided on the container.

80. The system of claim 79, wherein the temperature sensitive indicator comprises a temperature sensitive sticker.

81. The system of claim 80, wherein the temperature sensitive indicator provides a visual indication when the slurry composition reaches a pre-determined temperature.

82. The system of claim 81, wherein the pre-determined temperature is about −15° C.

83. The system of claim 81, wherein the pre-determined temperature is between about −19° C. and −11° C., between about −18° C. and −12° C., between about −17° C. and −13° C., or between about −16° C. and −14° C.

84. The system of any one of claims 79-83, wherein the temperature sensitive indicator provides a visual indication when the slurry composition is a colder temperature than the pre-determined temperature.

85. The system of any one of claims 79-84, wherein the temperature sensitive indicator provides a visual indication when the slurry composition is warmer than the pre-determined temperature.
